(19) United States
(12) Patent Application Publication
(10) Pub. No.: US 2022/0249385 A1
(43) Pub. Date: Aug. 11, 2022

(54) EXTENDED RELEASE COMPOSITION OF TOFACITINIB

(71) Applicant: Unichem Laboratories Limited, Mumbai, Maharashtra (IN)

(72) Inventors: Dhananjay SATHE, Maharashtra (IN); Bijay PADHI, Hyderabad Telengana (IN); Ankit BAHETI, Indore Madhya Pradesh (IN); Sachin BALI, Maharashtra (IN)

(73) Assignee: Unichem Laboratories Limited, Mumbai, Maharashtra (IN)

(21) Appl. No.: 17/440,124

(22) PCT Filed: Mar. 4, 2020
(86) PCT No.: PCT/IB2020/051829

§ 371 Date: Sep. 16, 2021

(30) Foreign Application Priority Data
Mar. 27, 2019 (IN) 201921012044

(51) Int. Cl. A61K 9/28; A61K 31/519; A61K 9/20

(52) U.S. Cl. CPC A61K 9/2846; A61K 31/519; A61K 9/28; A61K 9/2893; A61K 9/284; A61K 9/2866; A61K 9/2095; A61K 9/2853

(57) ABSTRACT

The present disclosure provides an extended release composition of tofacitinib for oral administration, and methods of making the composition. The extended release composition employs a matrix drug core comprising tofacitinib or a pharmaceutically acceptable salt thereof and at least one release controlling polymer. The matrix drug core is surrounded by a soluble functional coating comprising at least one coating polymer and at least one pharmaceutically acceptable excipient.

### FIELD OF THE INVENTION

The present disclosure relates to technical field of pharmaceutical formulation. In particular, the present disclosure relates to extended release formulation of tofacitinib for oral administration.

### BACKGROUND OF THE INVENTION

Tofacitinib citrate is a white to off-white powder. The chemical name of tofacitinib citrate is 3-{(3R,4R)-4-methyl-3-[methyl(7H-pyrrolo[2,3-d]pyrimidin-4-yl)amino]piperidin-1-yl}-3-oxo-propanenitrile,2-hydroxypropane-1,2,3-tricarboxylate. The solubility of tofacitinib citrate in water is 2.9 mg/ml. Tofacitinib citrate has a molecular weight of 504.5 Daltons (or 312.4 Daltons as the tofacitinib free base) and a molecular formula of C16H20N6O.C6H8O7. Tofacitinib citrate is disclosed in WO 02/096909 and U.S. Pat. No. 7,301,023, and its extended release product XEUANZ XR was approved in Feb. 23, 2016 by US FDA, which is used for the treatment of rheumatoid arthritis and psoriatic arthritis.

U.S. Pat. No. 9,937,181 discloses tofacitinib oral sustained release dosage forms, particularly osmotic dosage form of tofacitinib.

US Patent Application No. 20170049774A1 discloses sustained release composition of tofacitinib. Example 1 of this US patent application illustrates osmotic composition of tofacitinib, Example 2 illustrates matrix tablet, and Example 3 illustrates a reservoir tablet.

US Application No. 20170151244 discloses a dry process for preparation of tofacitinib tablets.

PCT Application No. 2014/174073 discloses a sustained release composition comprising tofacitinib, hydrophilic polymer and an alkalizing agent.

Previous research efforts and achievements notwithstanding, there exists a continuing need in the art for new and improved extended release formulation of tofacitinib for oral administration The present disclosure satisfies the existing needs, as well as others, and generally overcomes the deficiencies found in the prior art.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

### OBJECTS OF THE INVENTION

It is an object of the present disclosure to provide an extended release composition of tofacitinib or its pharmaceutically acceptable salt for oral administration.

It is another object of the present disclosure to provide a non-osmotic extended release dosage form of tofacitinib or its pharmaceutically acceptable salt.

It is another object of the present disclosure to provide an extended release composition of tofacitinib or its pharmaceutically acceptable salt that exhibits desired drug dissolution profile.

It is yet another object of the present disclosure to provide a simple and highly efficient process for preparation of extended release composition of tofacitinib or its pharmaceutically acceptable salt.

### SUMMARY OF THE INVENTION

Aspects of the present disclosure relate to an extended release composition of tofacitinib for oral administration, and methods of making the composition. The extended release composition employs a matrix drug core comprising tofacitinib or a pharmaceutically acceptable salt thereof and at least one release controlling polymer. The matrix drug core is surrounded by a soluble functional coating comprising at least one coating polymer and at least one pharmaceutically acceptable excipient. The functional coating surrounding the matrix drug core is water soluble and provides controlled porosity for drug release. Therefore, unlike osmotic pump dosage forms in the prior art, the extended release composition (e.g. tablet) of the present disclosure does not need to be drilled by laser to form an opening through the coating for drug release.

In one embodiment, tofacitinib or its pharmaceutically acceptable salt having a particle size distribution with a D90 of not more than 50 μm can be used in the extended release composition of the present disclosure.

In a preferred embodiment, tofacitinib or its pharmaceutically acceptable salt can have a particle size distribution with a D90 of not more than 40 μm.

In another aspect, the present disclosure provides a non-OROS (osmotic controlled release oral delivery system) method for preparation of the extended release composition of the present disclosure. Further the composition is core coated matrix system.

Various objects, features, aspects and advantages of the inventive subject matter will become more apparent from the following detailed description of preferred embodiments.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

FIG. 1 is a flow chart showing an exemplary process for producing extended release tablets of tofacitinib citrate in accordance with embodiments of the present disclosure.

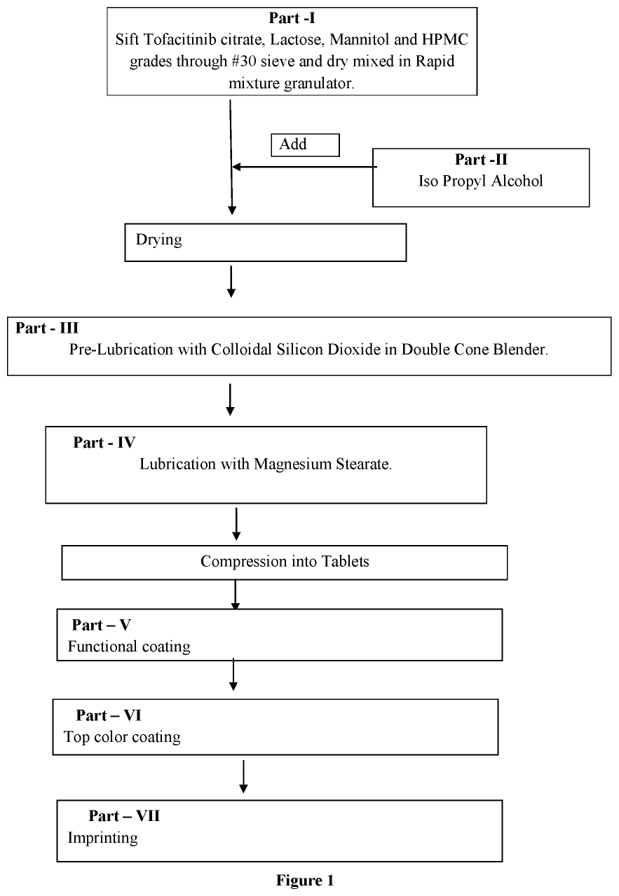

### DETAILED DESCRIPTION OF THE INVENTION

The following is a detailed description of embodiments of the present disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Unless the context requires otherwise, throughout the specification which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

In some embodiments, the numbers expressing quantities of ingredients, properties such as concentration, process conditions, and so forth, used to describe and claim certain embodiments of the invention are to be understood as being modified in some instances by the term “about.” Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

The headings and abstract of the invention provided herein are for convenience only and do not interpret the scope or meaning of the embodiments.

Various terms are used herein. To the extent a term used in a claim is not defined below, it should be given the broadest definition persons in the pertinent art have given that term as reflected in printed publications and issued patents at the time of filing.

As used herein the term “composition” includes but not limited to cores, coated cores, pellets, micro-pellets, pills, compressed tablets, granules, spheres, capsules and the like.

The term “tablet” is intended to encompass compressed pharmaceutical dosage forms of all shape and size, whether coated or uncoated.

The term “extended release tablet” as used herein refers to a mechanism that delivers a drug with a delay after its administration (delayed-release dosage) or for a prolonged period of time (extended-release [ER, XR, XL] dosage) or to a specific target in the body (targeted-release dosage). The term “OROS” means “Osmotic-Controlled Release Oral Delivery System’. The term PSD as used herein means ‘Particle Size Distribution’. The term RH as used herein means ‘Relative Humidity’. The term ASTM as used herein means ‘American Society for Testing and Materials’. The term μm as used herein means ‘micron’. The term RPM as used herein means ‘Revolutions/Rotations per Minutes’. The term LOD as used herein means ‘Loss on Drying.’ The term “NMT” as used herein means “Not More Than” The term “NLT” as used herein means “Not Less Than”.

The present disclosure provides an extended release composition of tofacitinib for oral administration, and methods of making the composition. In one aspect, the present disclosure provides an extended release composition comprising a therapeutically effective amount of tofacitinib or a pharmaceutically acceptable salt thereof in a matrix drug core, and a soluble functional coating surrounding the matrix drug core.

In one embodiment, the matrix drug core comprises tofacitinib or a pharmaceutically acceptable salt thereof and at least one release controlling polymer. In various embodiments, the matrix drug core can further include at least one diluent, at least one binder, at least one glidant and at least one lubricant.

The functional coating surrounding the matrix drug core comprises at least one coating polymer and at least one pharmaceutically acceptable excipient. In one embodiment, the at least one pharmaceutical excipient present in the functional coating can be any type of coating aid commonly known in the pharmaceutical art such as a plasticizer, an anti-tacking agent, solvents, a pore forming agent, a coloring agent or a combination thereof. According to embodiments of the present disclosure, the functional coating surrounding the matrix drug core is water soluble and provides controlled porosity for drug release. Therefore, unlike osmotic pump dosage forms in the prior art, the extended release composition (e.g. tablet) of the present disclosure does not need to be drilled by laser to form an opening through the coating for drug release.

In one embodiment, tofacitinib or its pharmaceutically acceptable salt having a particle size distribution with a D90 of not more than 50 μm can be used in the extended release composition of the present disclosure.

In a preferred embodiment, tofacitinib or its pharmaceutically acceptable salt can have a particle size distribution with a D90 of not more than 40 μm.

In a more preferred embodiment, tofacitinib or its pharmaceutically acceptable salt can have a particle size distribution with a D90 of not more than 20 μm.

Examples of the diluent used in the extended release composition of the present disclosure include, but not limited to, microcrystalline cellulose (MCC), microfine cellulose, powdered cellulose, lactose, dibasic calcium phosphate, tribasic calcium phosphate, starch, pre-gelatinized starch, calcium carbonate, calcium sulfate, magnesium carbonate, magnesium oxide, dextrates, dextrin, dextrose, kaolin, maltodextrin, mannitol, sucrose, methyl dextrin, sorbitol, and any combination thereof.

Examples of the binder used in the extended release composition of the present disclosure include, but not limited to, isopropyl alcohol, acetone, dimethyl formamide, ethanol, acetone, chloroform and the combination thereof, polyvinylpyrrolidone (povidone, PVP), polyethylene glycol (PEG), cross-linked polyvinylpyrrolidone, cellulose derivatives (such as hydroxymethyl cellulose, hydroxypropylcellulose (HPC), carboxy-methylcellulose sodium, ethyl cellulose, hydroxylethylcellose, hydroxypropylmethylcellulose), sucrose, alginic acid or sodium alginate, carbomer, cottonseed oil, dextrin, dextrose, guar gum, hydrogenated vegetable oil type I, magnesium aluminium silicate, maltodextrin, maltose, polydextrose, polyethylene oxide, stearic acid, zein and any combination thereof.

The release controlling polymer used in the matrix drug core includes water soluble polymers and water insoluble polymers. The water soluble polymers may preferably be selected from polyvinylpyrrolidone, polyethylene oxide, polyethylene oxide, polymers of acrylic and methacrylic acids and esters thereof, cellulose derivatives such as hydroxypropylmethylcellulose, hydroxymethyl cellulose, hydroxypropyl methylcellulose and any combination thereof. The more preferably water swellable polymers selected from the group consisting of water soluble polymers. Preferred water insoluble polymers include ethylcellulose, cellulose acetate, cellulose acetate phthalate, polymethyacrylates, calcium silicates, sodium alginate, and any combination thereof.

Examples of the lubricant used in the extended release composition of the present disclosure include, but not limited to, magnesium stearate, aluminium stearate, sucrose stearate, stearic acid, talc, fumaric acid, palmitic acid, sodium stearyl fumarate, glyceryl monostearate, caranuba wax, hydrogenated vegetable oils, mineral oil, polyethylene glycols and any combination thereof.

Examples of glidant include, but not limited to, colloidal silicon dioxide and derivatives thereof.

The coating polymer present in the functional coating can be selected from the group consisting of alkyl celluloses, hydroxyalkyl celluloses, ethyl cellulose, cellulose ethers, cellulose esters, nitro celluloses, polymers of acrylic and methacrylic acids and esters thereof, polyamides, polycarbonates, polyalkylenes, polyalkylene glycols, polyalkylene oxides, polyalkylene terephthalates, polyvinyl alcohols, polyvinyl ethers, polyvinyl esters, cellulose acetate, cellulose acetate butyrate, agar acetate, amylose triacetate, poly (vinyl methyl) ether copolymers, poly (orthoesters), polyacetals, poly (glycolic acid), poly (lactic acid), derivatives thereof, co-polymers thereof and a combination thereof. Preferably, the coating polymer is selected from one or more of hydroxypropylmethyl cellulose (HPMC), hydroxypropyl cellulose, hydroxyethyl cellulose, polymers of acrylic and methacrylic acids and esters thereof.

Suitable plasticizer includes, but not limited to, triethyl citrate, triacetin, acetylated monoglyceride, diethyl phthalate, hydroxypropylmethylcellulose acetate succinate, polyethylene glycol 6000, tributyl citrate, dibutyl sebacate and any combination thereof.

The coloring agent can be any FDA approved color for oral use. Preferred coloring agent includes Instacoat, food colors such as FD&C colors such as FD&C Yellow No. 6, FD&C Red No. 2, FD&C Blue No. 2, food lakes and the like.

According to embodiments of the present disclosure, the disclosed extended release composition can be formulated into tablet, capsule, powder, disc, caplet, granules, pellets, tablet in tablet, tablet in capsule, pellets in capsule, powder in capsule, granules in capsule and other dosage forms suitable for oral administration. Tablets may further be coated with film forming polymers.

In another aspect, the present disclosure provides a process for preparation of the extended release composition of the present disclosure. In an embodiment, the extended release composition of the present disclosure can be prepared by wet granulation method.

In one exemplary embodiment, the disclosed extended release composition can be formulated into extended release tablets by using the following method:
a) Dry Mixing: Sifting tofacitinib citrate, lactose, mannitol and release controlling polymer through #30 sieve and dry mixing in rapid mixture granulator for 10 minutes.
b) Binder addition: Spraying isopropyl alcohol on the dry mix obtained in step a).
c) Drying and sizing: Drying the granules in fluidized bed dryer for 30 minutes and sizing by using #30 sieve.
d) Pre-lubrication: Pre-lubricating the sized granules by using colloidal silicon dioxide (Aerosil 200) in a double cone blender for 5 minutes.
e) Lubrication: Lubricating the pre-lubricated granules by using magnesium stearate in a double cone blender for 3 minutes.
f) Compression: Compressing the blend obtained in e) into tablets by using appropriate punch set.
g) Functional Coating: Providing the functional coating on the outer surface of the compressed tablets.
h) Top color coating: Providing a color coating on the functional coated tablets.
i) Imprinting: Imprinting the color coated tablets using imprinting ink solution.

An extended release oral pharmaceutical composition of tofacitinib may comprise from about 10 mg to about 12 mg of tofacitinib, preferably 11 mg of tofacitinib. Further, the pharmaceutical composition may comprise from about 20 to about 24 mg of tofacitinib, preferably 22 mg of tofacitinib.

While the foregoing description discloses various embodiments of the disclosure, other and further embodiments of the disclosure may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

The present disclosure is further explained in the form of following examples. However, it is to be understood that the foregoing examples are merely illustrative and are not to be taken as limitations upon the scope of the invention. Various changes and modifications to the disclosed embodiments will be apparent to those skilled in the art. Such changes and modifications may be made without departing from the scope of the invention. Following are the examples for 11 mg & 22 mg of tofacitinib tablets.

### Example-1

| | | | |
|---|---|---|---|
| | | Composition | |
| Sr. No | Ingredient | % w/w | Function |

Part-I Dry mix

| | | | |
|---|---|---|---|
| 1 | Tofacitinib Citrate | 8.19 | Active |
| 2 | Lactose monohydrate | 30.47 | Diluent |
| 3 | Mannitol | 30.47 | Diluent |
| 4 | Hyprornellose 2208 | 20.74 | Control release polymer |

Part-II (Binder solution)

| | | | |
|---|---|---|---|
| 5 | Iso Propyl Alcohol | q.s | Binder |

Part-III (Pre-lubrication)

| | | | |
|---|---|---|---|
| 6 | Colloidal Silicon Dioxide | 0.46 | Glidant |

Part-IV (Lubrication)

| | | | |
|---|---|---|---|
| 7 | Mamestum stearate | 1.84 | Lubricant |

| | |
|---|---|
| Total weight of core tablet | 92.17 |

Part-V (Functional Coating)

| | | | |
|---|---|---|---|
| 8 | Poly(methacrylic | 1.58 | Coating polymer |
| | acid-co-ethyl acrylate) 1:1 | | |
| 9 | Hypromellose 2910 | 2.37 | Coating polymer |
| 10 | Tri Ethyl Citrate | 0.39 | Plasticizer |
| 11 | Talc (Micronised) | 1.18 | Anti-tacking agent |
| 12 | Acetone | q.s | Coating solvent |
| 13 | Iso Propyl Alcohol | q.s | Coating solvent |
| 14 | Purified water | q.s | Coating solvent |

| | |
|---|---|
| Total weight of functional coated tablet | 97.7 |

Part-VI (Top color coating)

| | | | |
|---|---|---|---|
| 15 | Instacoat Uni. Pink | 2.30 | Coating agent |
| 16 | Purified Water | q.s | Coating solvent |

| | |
|---|---|
| Total weight of color coated tablet | 100 |

Part-VII (Imprinting)

| | | | |
|---|---|---|---|
| 17 | Opacode Black | q.s | Imprinting agent |
| 18 | Iso Propyl Alcohol | q.s | Imprinting solvent |

| | |
|---|---|
| Total weight of finished tablet | 100 |

TABLE 1
Core Tablet Dissolution Results of Example 1

| Dissolution Media | Time (Hours) | Example 1 |
|---|---|---|
| Medium: | 0 | 0 |
| pH 6.8 phosphate | 0.5 | 28 |
| buffer | 1 | 46 |
| USP Apparatus: | 1.5 | 61 |
| Paddle | 2 | 76 |
| Volume: 900 ml | 2.5 | 88 |
| RPM: 50 rpm | 3 | 95 |
| | 4 | 99 |
| | 6 | 100 |
| | Infinity | 101 |

TABLE 2
Coated Tablet Dissolution Results of Example 1

| Dissolution Media | Time (Hours) | Example 1 |
|---|---|---|
| Medium: | 0 | 0 |
| pH 6.8 phosphate | 0.5 | 14 |
| buffer | 1 | 30 |
| USP Apparatus: | 1.5 | 44 |
| Paddle | 2 | 57 |
| Volume: 900 ml | 2.5 | 69 |
| RPM:50 rpm | 3 | 79 |
| | 4 | 96 |
| | 6 | 99 |
| | Infinity | 100 |

### Example-2

| | | | |
|---|---|---|---|
| | | Composition | |
| Sr. No | Ingredient | % w/w | Function |

Part-I (Dry mix)

| | | | |
|---|---|---|---|
| 1 | Tofacitinib Citrate | 8.19 | Active |
| 2 | Lactose monohydrate | 22.40 | Diluent |
| 3 | Mannitol | 22.40 | Diluent |
| 4 | Hypromellose 2208 | 20.74 | Control release polymer |
| 5 | Hypromellose 2910 | 16.13 | Control release polymer |

Part-II (Binder solution)

| | | | |
|---|---|---|---|
| 6 | Iso Propyl Alcohol | q.s | Binder |

Part-III (Pre-lubrication

| | | | |
|---|---|---|---|
| 7 | Colloidal Silicon Dioxide | 0.46 | aid+32 |

Part-IV (Lubrication)

| | | | |
|---|---|---|---|
| 8 | Magnesium stearate | 1.84 | Lubricant |

| | |
|---|---|
| Total weight of core tablet | 92.17 |

Part-V (FunctionalCoating)

| | | | |
|---|---|---|---|
| 9 | Poly(methacrylic | 1.58 | Coating polymer |
| | acid-co-ethyl acrylate) 1:1 | | |
| 10 | Hypromellose 2910 | 2.37 | Coating polymer |
| 11 | Tri Ethyl Citrate | 0.39 | Plasticizer |
| 12 | Talc (Micronised) | 1.18 | Anti-tacking agent |
| 13 | Acetone | q.s | Coating solvent |
| 14 | Iso Propyl Alcohol | q.s | Coating solvent |
| 15 | Purified water | q.s | Coating solvent |

| | |
|---|---|
| Total weight of functional coated tablet | 97.7 |

Part-VI (Top color coating)

| | | | |
|---|---|---|---|
| 16 | Instacoat Uni. Pink | 2.3 | Coating agent |
| 17 | Purified Water | q.s | Coating solvent |

| | |
|---|---|
| Total weight of color coated tablet | 100 |

Part-VII (Imprinting)

| | | | |
|---|---|---|---|
| 18 | Opacode Black | q.s | Imprinting agent |
| 19 | Iso Propyl Alcohol | q.s | Imprinting solvent |

| | |
|---|---|
| Total weight of finished tablet | 100 |

TABLE 3
Core Tablet Dissolution Results of Example 2

| Dissolution Media | Time (Hours) | Example 2 |
|---|---|---|
| Medium: | 0 | 0 |
| pH 6.8 phosphate | 0.5 | 20 |
| buffer | 1 | 31 |
| USP Apparatus: | 1.5 | 42 |
| Paddle | 2 | 50 |
| Volume: 900 ml | 2.5 | 58 |
| RPM: 50 rpm | 3 | 66 |
| | 4 | 80 |
| | 6 | 96 |
| | Infinity | 100 |

TABLE 4
Coated Tablet Dissolution Results of Example 2

| Dissolution Media | Time (Hours) | Example 2 |
|---|---|---|
| Medium: | 0 | 0 |
| pH 6.8 phosphate | 0.5 | 13 |
| buffer | 1 | 24 |
| USP Apparatus: | 1.5 | 34 |
| Paddle | 2 | 44 |
| Volume: 900 ml | 2.5 | 52 |
| RPM: 50 rpm | 3 | 60 |
| | 4 | 74 |
| | 6 | 93 |
| | Infinity | 101 |

### Example-3

| | | | |
|---|---|---|---|
| | | Composition | |
| Sr. No | Ingredient | % w/w | Function |

Intragranular

| | | | |
|---|---|---|---|
| 1 | Tofacitinib Citrate | 8.19 | Active |
| 2 | Lactose monohydrate | 30.47 | Diluent |
| 3 | Mannitol | 30.47 | Diluent |
| 4 | Hypromellose 2208 | 20.74 | Control release polymer |

Binder solution

| | | | |
|---|---|---|---|
| 5 | Iso Propyl Alcohol | q.s | Binder |

Prelubrication

| | | | |
|---|---|---|---|
| 6 | Colloidal Silicon Dioxide | 0.46 | Glidant |

Lubrication

| | | | |
|---|---|---|---|
| 7 | Magnesium stearate | 1.84 | Lubricant |

| | |
|---|---|
| Total weight of core tablet | 92.17 |

Functional Coating

| | | | |
|---|---|---|---|
| 8 | Ethyl cellulose std. 7 premium. | 2.76 | Coating polymer |
| 9 | Hypromellose 2910 | 1.19 | Coating polymer |
| 10 | Tri Ethyl Citrate | 0.39 | Plasticizer |
| 11 | Talc (Micronized) | 1.18 | Anti-tacking agent |
| 12 | Iso Propyl Alcohol | q.s | Coating solvent |
| 13 | Purified water | q.s | Coating solvent |

| | |
|---|---|
| Total weight of functional coated tablet | 97.7 |

Top color coating

| | | | |
|---|---|---|---|
| 14 | Instacoat Uni. Pink | 2.3 | Coating agent |
| 15 | Purified Water | q.s | Coating solvent |

| | |
|---|---|
| Total weight of color coated tablet | 100 |

Imprinting

| | | | |
|---|---|---|---|
| 16 | OPACODE BLACK | q.s | imprinting agent |
| 17 | Iso Propyl Alcohol | q.s | Imprinting solvent |

| | |
|---|---|
| Total weight of finished tablet | 100 |

TABLE 5
Dissolution Results of Example 3

| Dissolution Media | Time (Hours) | Example 3 |
|---|---|---|
| Medium: | 0 | 0 |
| pH 6.8 phosphate | 0.5 | Not Done |
| buffer | 1 | 18 |
| USP Apparatus: | 1.5 | 30 |
| Paddle | 2 | 40 |
| Volume: 900 ml | 2.5 | 55 |
| RPM: 50 rpm | 3 | 80 |
| | 4 | 89 |
| | 6 | 95 |
| | Infinity | 96 |

### Example-4

| | | Composition | |
|---|---|---|---|
| Sr. | Ingredient | % w/w | Function |
| | Core composition | | |
| | Dry mix | | |
| 1 | Tofacitinib Citrate | 8.89 | Active |
| 2 | Lactose monohydrate | 22.10 | Diluent |
| 3 | Mannitol | 22.10 | Diluent |
| 4 | Hypromellose 2208 | 45.45 | Control release polymer |
| | Binder | | |
| 5 | Iso Propyl Alcohol | q.s. | Solvent |
| | Pre-lubrication | | |
| 6 | Colloidal Silicon Dioxide | 0.45 | Glidant |
| | Lubrication | | |
| 7 | Magnesium stearate | 1.82 | Lubricant |
| | Core tablet weight | 100 | |

### Example 5

| | | | |
|---|---|---|---|
| | | Composition | |
| Sr. No | Ingredient | % w/w | Function |

Part I-(Dry mix)

| | | | |
|---|---|---|---|
| 1 | Tofacitinib Citrate | 8.19 | Active |
| 2 | Lactose monohydrate | 28.16 | Diluent |
| 3 | Mannitol | 28.16 | Diluent |
| 4 | Hypromellose 2208 | 11.52 | Control release |
| | | | polymer |
| 5 | Hypromellose 2208 | 13.82 | Control release |
| | | | polymer |

Part II-(Binder solution)

| | | |
|---|---|---|
| 6 | Isopropyl Alcohol | Binder |

Part III-(Prelubrication)

| | | | |
|---|---|---|---|
| 7 | Colloidal Silicon Dioxide | 0.46 | Glidant |

Part IV-(Lubrication)

| | | | |
|---|---|---|---|
| 8 | Magnesium Stearate | 1.84 | Lubricant |

| | |
|---|---|
| Percent total weight of core tablet | 92.17 |

Part V-Functional coating

| | | | |
|---|---|---|---|
| 9 | Poly(methacrylic | 1.58 | Coating polymer |
| | acid-co-ethylacrylate) 1:1 | | |
| 10 | Hypromellose 2910 | 2.37 | Coating polymer |
| 11 | Triethylcitrate | 0.39 | Plasticizer |
| 12 | Talc (Micronised) | 1.18 | Anti-tacking agent |
| 13 | Acetone | q.s. | Coating solvent |
| 14 | IPA | q.s. | Coating solvent |
| 15 | Purified water | q.s. | Coating solvent |

| | |
|---|---|
| Percent total weight of functional coated tablet | 97.7 |

Part VI-(Top color coating)

| | | | |
|---|---|---|---|
| 16 | Instacoat Uni. Pink | 2.30 | Coating agent |
| 17 | Purified water | q.s. | Coating solvent |

| | |
|---|---|
| Percent total weight of color coated tablet | 100 |

Part VII-(Imprinting)

| | | | |
|---|---|---|---|
| 18 | OPACODE BLACK | q.s. | Imprinting agent |
| 19 | Isopropyl Alcohol | q.s. | Imprinting solvent |

| | |
|---|---|
| Percent total weight of finished tablet | 100 |

### Example 6

| | | | |
|---|---|---|---|
| | | Composition | |
| Sr. No | Ingredient | % w/w | Function |

Part I-(Dry mix)

| | | | |
|---|---|---|---|
| 1 | Tofacitinib Citrate | 8.19 | Active |
| 2 | Lactose monohydrate | 21.25 | Diluent |
| 3 | Mannitol | 21.25 | Diluent |
| 4 | Hypromellose 2208 | 39.17 | Control release |
| | | | polymer |

Part II-(Binder solution)

| | | | |
|---|---|---|---|
| 5 | Isopropyl Alcohol | q.s. | Binder |

Part III-(Prelubrication)

| | | | |
|---|---|---|---|
| 6 | Aerosil | 0.46 | Glidant |

Part IV-(Lubrication)

| | | | |
|---|---|---|---|
| 7 | Magnesium Stearate | 1.84 | Lubricant |

| | |
|---|---|
| Percent total weight of core tablet | 92.17 |

Part V-Functional coating

| | | | |
|---|---|---|---|
| 8 | Poly(methacrylic | 1.58 | Coating polymer |
| | acid-co-ethylacrylate) 1:1 | | |
| 9 | Hypromellose 2910 | 2.37 | Coating polymer |
| 10 | Triethylcitrate | 0.39 | Plasticizer |
| 11 | Talc (Micronised) | 1.18 | Anti-tacking agent |
| 12 | Acetone | q.s. | Coating solvent |
| 13 | IPA | q.s. | Coating solvent |
| 14 | Purified water | q.s. | Coating solvent |

| | |
|---|---|
| Percent total weight of functional coated tablet | 97.7 |

Part VI-(Top color coating)

| | | | |
|---|---|---|---|
| 15 | Instacoat Uni. Pink | 2.30 | Coating agent |
| 16 | Purified water | q.s | Coating solvent |

| | |
|---|---|
| Percent total weight of color coated tablet | 100 |

Part VII-(Imprinting)

| | | | |
|---|---|---|---|
| 17 | OPACODE BLACK | q.s. | Imprinting agent |
| 18 | Isopropyl Alcohol | q.s. | Imprinting solvent |

| | |
|---|---|
| Total weight of finished tablet | 100 |

TABLE 6
Swelling and Erosion Study

| | Swelling | % Water | % Erosion | % Erosion |
|---|---|---|---|---|
| Example | Index | uptake | at 45 min | at 75 min |
| Example 1 | 0.15 | 15.41 | 44.73 | 58.18 |
| Example 2 | 0.18 | 18.06 | 37.99 | 57.53 |
| Example 5 | 1.1 | 105.02 | 38.07 | 44.80 |
| Example 6 | 0.80 | 78.54 | 33.18 | 44.80 |

TABLE 7
Comparative data of % Hydrophilic content
(Non-polymer) and % Polymer content

| | % Hydrophilic | |
|---|---|---|
| | content | % Polymer |
| Example | (non-polymer) | content |
| Example 1 | 66.12 | 22.5 |
| Example 2 | 48.62 | 40 |
| Example 5 | 61.12 | 27.5 |
| Example 6 | 46.12 | 42.5 |

Swelling Study and Erosion Study

Tablets were placed in disintegration test apparatus containing 0.05 M phosphate buffer, pH 6.8 at 37° C. The rate of uptake of the medium by the tablets and the rate of polymer erosion were determined by gravimetric analysis methods. Dry matrix tablets were accurately weighed using a precision weighing balance. In separate experiments, tablets were removed from the dissolution medium at the interval of 15 minutes following exposure to the dissolution medium and lightly blotted with filter paper to remove excess water. The swollen tablets were weighed to determine the extent of water uptake and then dried in a convection oven at 60° C. After 12 h, the tablets were cooled to ambient temperature and then weighed. This measurement was termed the dried weight. The percent increase in tablet weight that can be attributed to the uptake or absorption of water was calculated at each time point using Equation 1. The percent matrix erosion was estimated at each time point using Equation 2

$$\begin{matrix}Percent Swelling = \frac{{W}_{s} - {W}_{i}}{{W}_{i}} \times 100 & Equation 1 \\ Percent matrix erosion = \frac{{W}_{i} - {W}_{t}}{{W}_{t}} \times 100 & Equation 2\end{matrix}$$

where Ws is the weight of swollen samples at sampling times,
Wi is the initial weight of tablet, and
Wt is the weight of dried matrices at sampling times.

We claim:

1. An extended release oral pharmaceutical composition comprising tofacitinib or its pharmaceutically acceptable salt thereof, a control release polymer, and pharmaceutically acceptable excipients, wherein composition is core coated matrix system.

2. The extended release oral pharmaceutical composition as claimed in claim 1, tofacitinib pharmaceutically acceptable salt thereof is tofacitinib citrate.

3. The extended release oral pharmaceutical composition as claimed in claim 1, wherein the composition is an extended release matrix tablet.

4. The extended release oral pharmaceutical composition as claimed in claim 1, wherein composition further comprises an outer functional coating and/or top coating which comprises water soluble polymer.

5. The extended release oral pharmaceutical composition as claimed in claim 4, wherein the water soluble polymer is selected from the group consisting of but not limited to such as polyvinylpyrrolidone, polyethylene oxide, polymers of acrylic and methacrylic acids and esters thereof, cellulose derivatives such as hydroxypropyl methylcellulose, hydroxymethyl cellulose or hydroxypropyl methylcellulose or any combination thereof.

6. The extended release oral pharmaceutical composition as claimed in claim 1, wherein the control release polymer is water soluble polymer.

7. An extended release oral pharmaceutical composition comprising of tofacitinib citrate, wherein D90 value of the particle size distribution of the tofacitinib citrate is NMT 50 μm.

8. The extended release oral pharmaceutical composition as claimed in claim 3, wherein the extended release matrix tablet comprises:
(i) a core comprising tofacitinib or a pharmaceutically acceptable salt thereof, a diluent, a control release polymer, and other pharmaceutically acceptable excipients;
(ii) a functional coating over the core, wherein the functional coating comprises a coating polymer, plasticizer, and a coating solvent; and
(iii) optionally, the top coating over the functional coating.

9. The extended release oral pharmaceutical composition as claimed in claim 1, wherein the composition has dissolution profile of not more than 31% of the tofacitinib, or pharmaceutically acceptable salt thereof, in 1 hour, not less than 40% of the tofacitinib, or pharmaceutically acceptable salt thereof, in 4 hours and not less than 75% of the tofacitinib, or pharmaceutically acceptable salt thereof, in 8 hours and wherein said extended release dosage form of tofacitinib, or pharmaceutically acceptable salt thereof, completely dissolves within 10-12 hours in a test medium comprising 900 ml of 0.05M pH 6.8 potassium phosphate buffer at 37° C. and a standard USP rotating paddle apparatus and the paddles are rotated at 50 rpm.

10. A process for the preparation of pharmaceutical composition comprising of tofacitinib or a pharmaceutical salt thereof; wherein the process comprising the steps of dry mixing of tofacitinib or pharmaceutically acceptable salt thereof, and pharmaceutically acceptable excipients,
spraying a binder on dry mixed excipients,
drying and sizing of granules,
prelubricating & lubricating of sized granules,
compressing of blend into the tablets,
functional coating of compressed tablets by using functional coating solution, and optionally, top coating over functional coated tablets.

11. The extended release oral pharmaceutical composition as claimed in claim 1, wherein said pharmaceutical composition has swelling index NMT 2 and/or erosion at 75 min. NLT 10%.
